(19) United States
(12) Patent Application Publication
(10) Pub. No.: US 2006/0013906 A1
(43) Pub. Date: Jan. 19, 2006

(54) Anti-helicobacter activity of celery seed extract

(76) Inventors: Kim Drummond Rainsford, Baslow (GB); Zhong-Ping Liu, Sheffield (GB)

Correspondence Address: ICE MILLER, ONE AMERICAN SQUARE, BOX 82001, INDIANAPOLIS, IN, 46282-0200, US

(21) Appl. No.: 10/515,985

(22) PCT Filed: May 22, 2003
(86) PCT No.: PCT/GB03/02244

§ 371 Date: Jun. 22, 2005

(30) Foreign Application Priority Data
May 23, 2002 (GB) 0211943.6

(51) Int. Cl. A61K 36/23

(52) U.S. Cl. 424/776

(57) ABSTRACT

The application discloses celery seeds or celery seed extract for treating Helicobacter pylori infections.

The inventor relates to the use of biologically active celery seed extracts to inhibit the growth and replication of the bacterium, Helicobacter pylori.

Arthritis and rheumatism are important world-wide problems, Around 1% of the UK population are affected at some stage in life. Complaints of this nature not only cause significant disability but may also have a severely detrimental effect on the psychological state of the sufferers. Conventionally these complaints are treated with analgesic/antipyretic drugs and non-steroid anti-inflammatory drugs (NSAIDs) However NSAIDs can have serious side effects, such as gastotoxicity, causing for example gatric ulceration, and hence research has been made into alternative sources of anti-inflammatory drugs. In particular compounds extracted from higher plants have been considered. Lewis et al (1985) and Whitehouse et al (1999) found that the extracts of celery (Apium graveolens) (CSE) had significant anti-inflammatory activity in animal models with reduced adverse effects A further risk factor in the pathogenesis of peptic ulcer disease is H. pylori infection. Chan (1997) found that eradication of H. pylori before NSAID therapy reduced the risk of ulcer development by about fourfold PCT/US99/25873 discloses the use of celery seed extract for the prevention and treatment of pain inflammation and gastrointestinal irritation.

The inventors have surprisingly found that components of celery seed extract may be used to control the growth of Helicobacter pylori.

The invention provides the use of celery seed or celery seed extract (CSE) for the inhibition of growth and replication of Helicobacter pylori

A preferred CSE is produced by supercritical fluid extraction of the starting product, By CSE we mean a natural product derived from celery seed, or a pharmaceutical equivalent thereof. This is preferably an ethanol/water extract, especially 50% to 90%, 60% to ,5%. most preferably an 80% Vol:Vol ethanol/water extract. The term includes the isolated compounds obtainable from CSE.

Preferably the active component of the celery seed extract is selected from the group: 3-n-butyl 4,5-dihydrolphtlide, 3-n-butyl phthalide, α-Budesmol, β-Budesmol dioctyl phthalate and cis, cis-9,1 2-Octadecadienoic acid.

The invention further provides a phannaceutical composition for the inhibition of growth and replication of Helicobacter pylori, comprising celery seed extract.

Also provided is the use of celery seed or celery seed extract in the preparation of a pharmaceutical composition for the treatment of Helicobacter pylori infection.

Preferably the H. pylori infection is in a al, such as a human. Preferably the infection is within the digestive tract, especially the stomach of the mammal.

The pharmaceutical composition may be administered orally, e.g. in the form of an oral suspension, solution or tablet. Dosages may be 300-2000 mg. daily in divided doses preferably or even higher.

The pharmaceutical composition may comprise one or more pharmaceutically acceptable carriers, bulking agents or excipients known in the art (e.g. in the form of a tablet or injectable solution).

A further aspect of the invention provides celery seed or celery seed extract for use in the manufacture of a medicament to treat a Helicobacter pylori infection.

The invention will now be described in detail with reference to the figures in which:

Table 1 shows the effect of the crude extract of CSE on the growth of different strains (3330, 3336 and 3339) of H. pylori.

Table 2 shows the distribution of antimicrobial activity against H. pylori (strain 3339) in the crude extract and different fractions of CSE.

Table 3 shows antimicrobial activity of the subtractions from pet. ether fraction against H. pylori (strain 3339).

Table 4 shows antimicrobial activities of compounds from subfractions 6 and 10 against H. pylori (strain 3339).

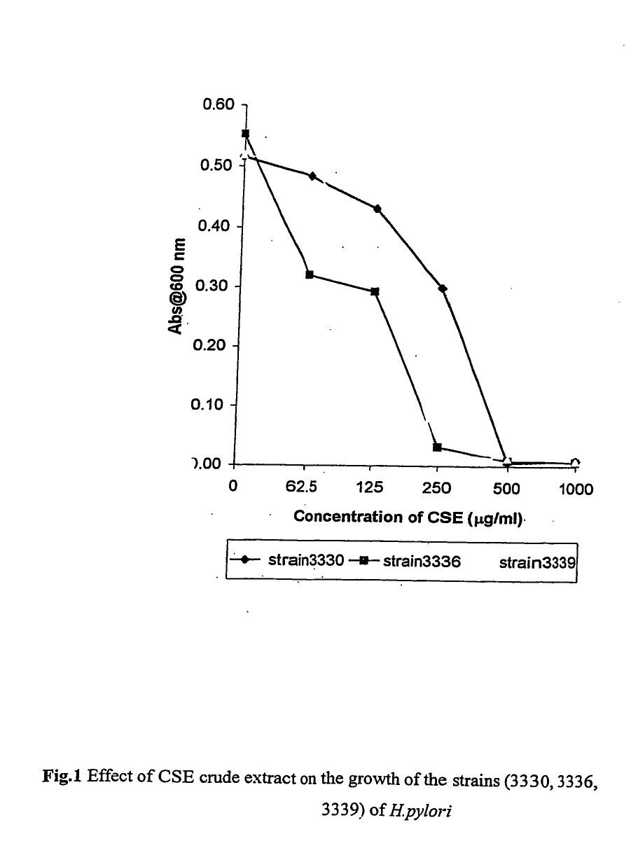

FIG. 1 shows the effect of CSE crude extract on the growth of the strains (3330, 3336, 3339) of H. pylori

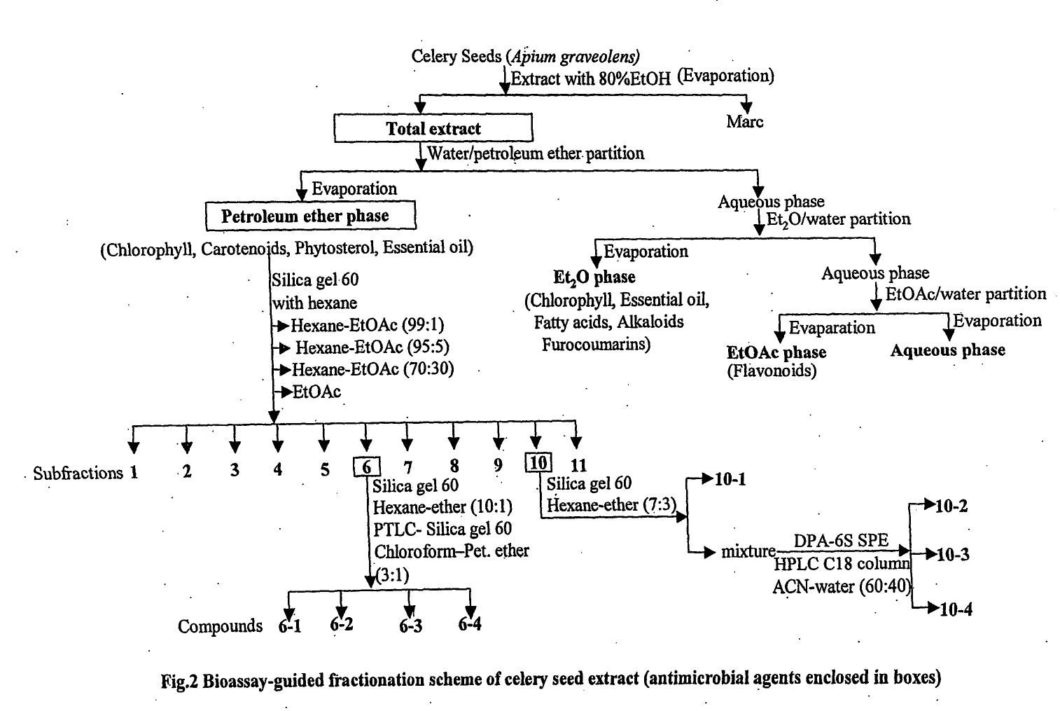

FIG. 2 shows the bioassay-guided frationation scheme of celery seed extract (antimicrobial agents enclosed in boxes).

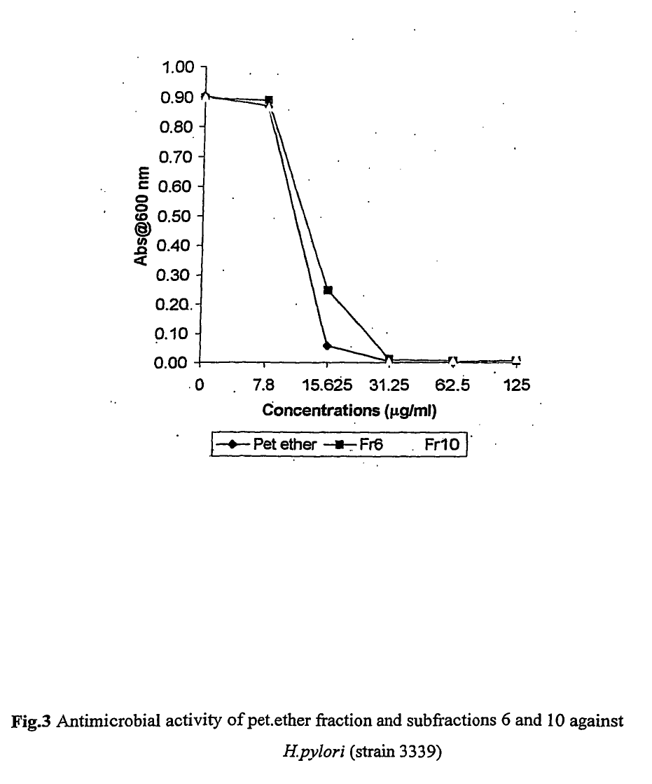

FIG. 3 shows the antimicrobial activity of pet. ether fraction and subfractions 6 and 10 against H. pylori (strain 3339).

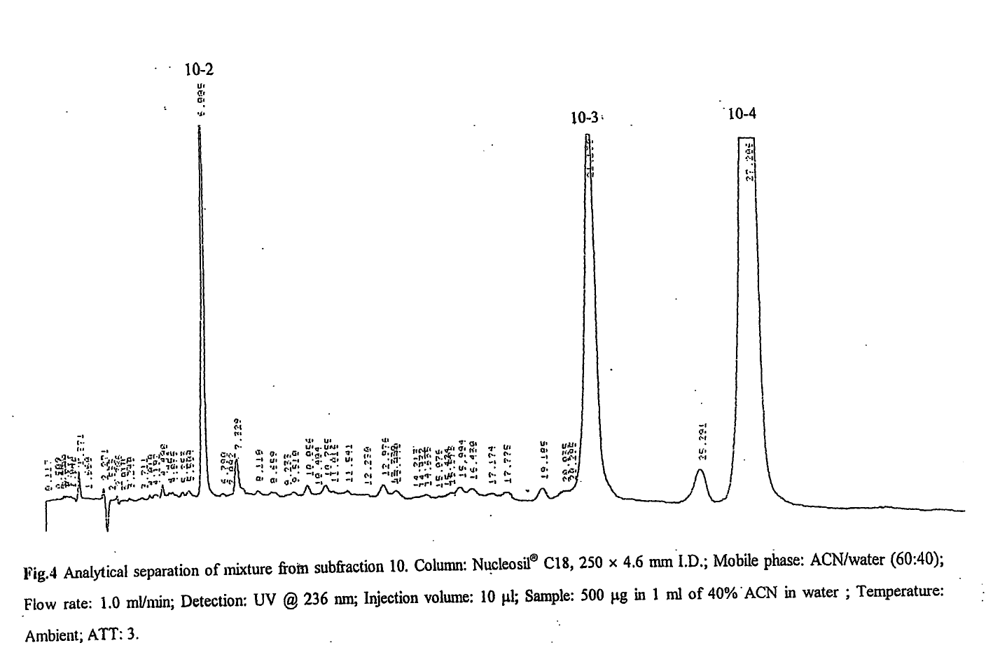

FIG. 4 shows the analytical separation of mixture from subfraction 10. Column: Nucleosil® C18, 250×4.6 mm. I.D.; Mobilo phase: ACN/water (60:40); Flow ram: 1.0 m./min; Detection: WV @ 236 nm; Injection volume: 10 jig in 1 ml of 40% ACN in water; Temperature: Ambient; ATT.3.

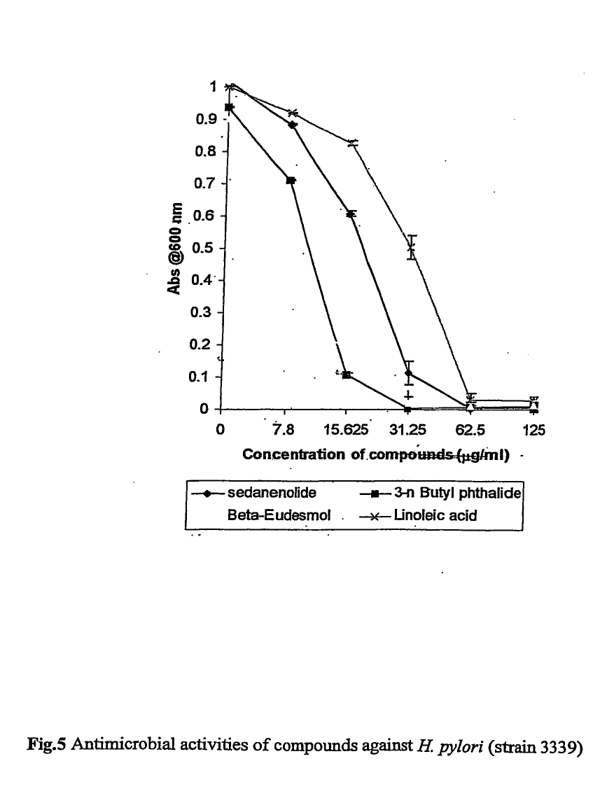

FIG. 5 shows the antimicrobial activities of compounds against H. pylori (strain 3339)

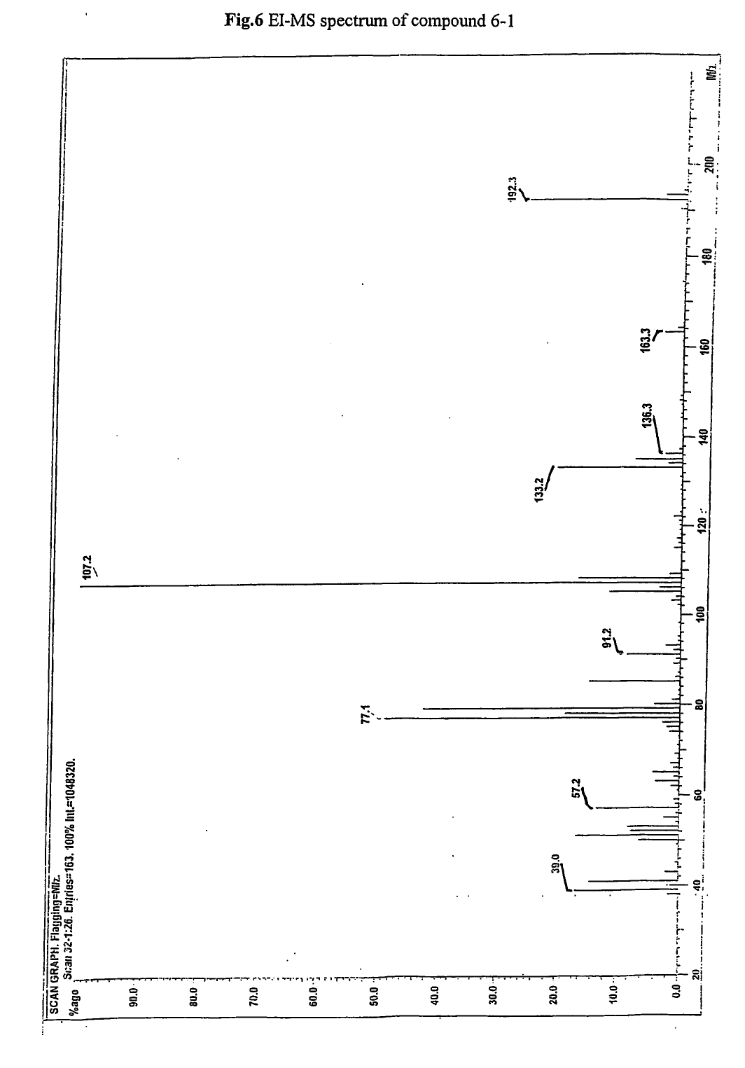

FIG. 6 shows the EI-MS spectrum of compound 6-1

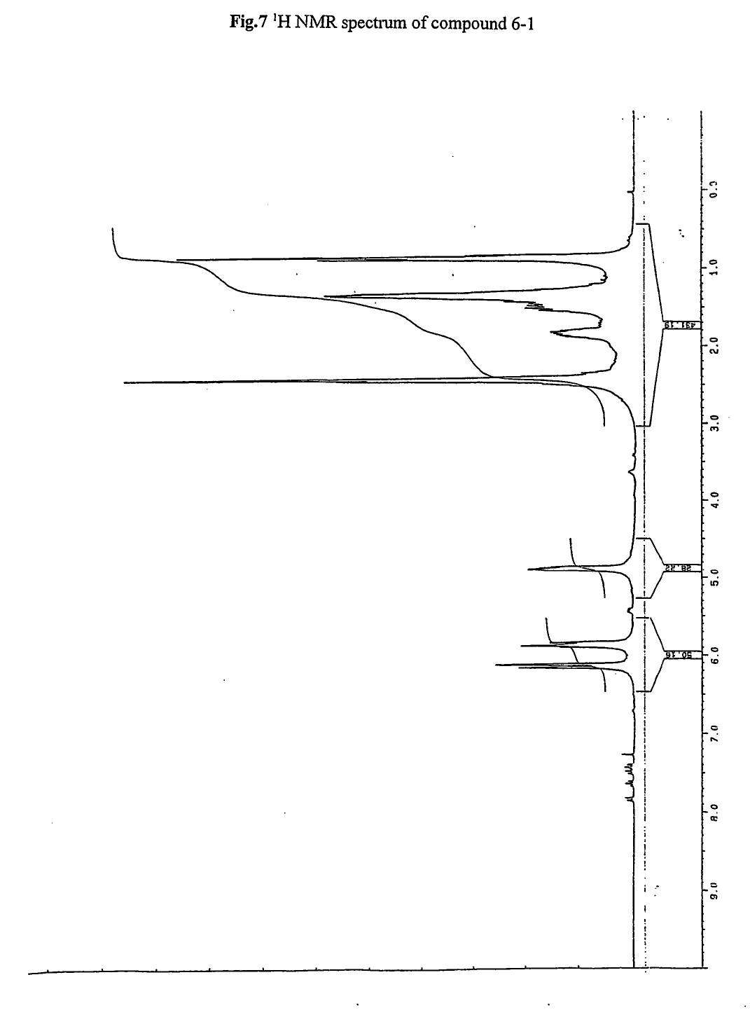

FIG. 7 shows the ¹H NMR spectrum of compound 6-1

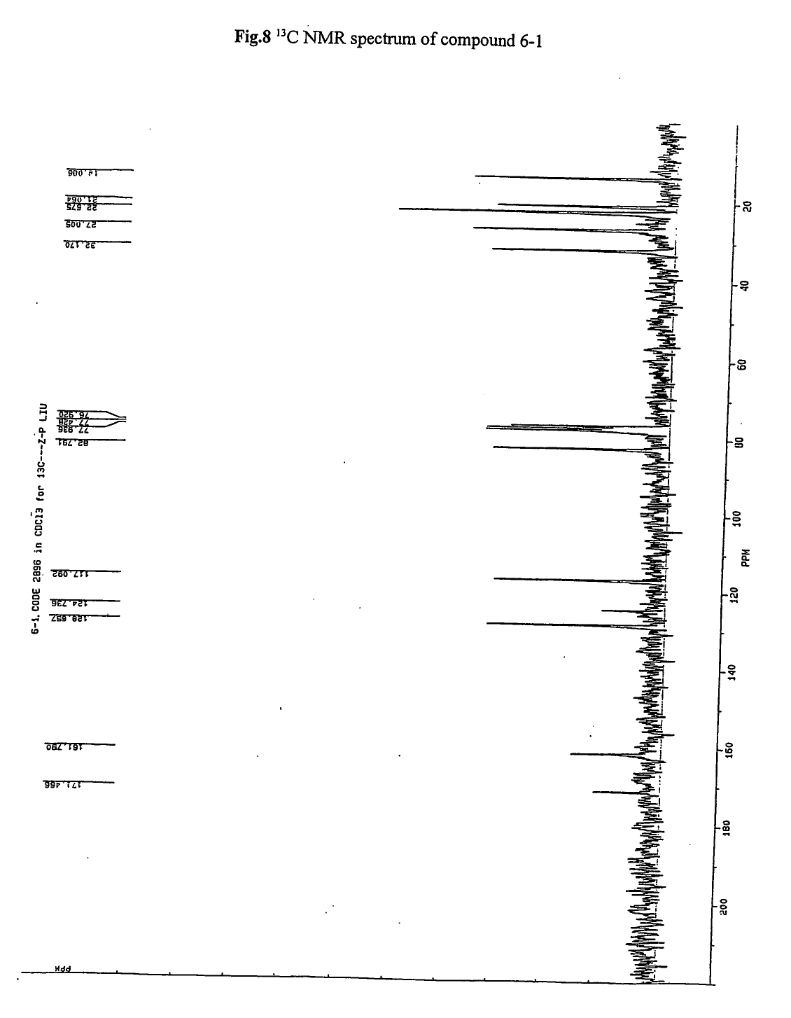

FIG. 8 shows the ¹³C NM spectrum of compound 6-1

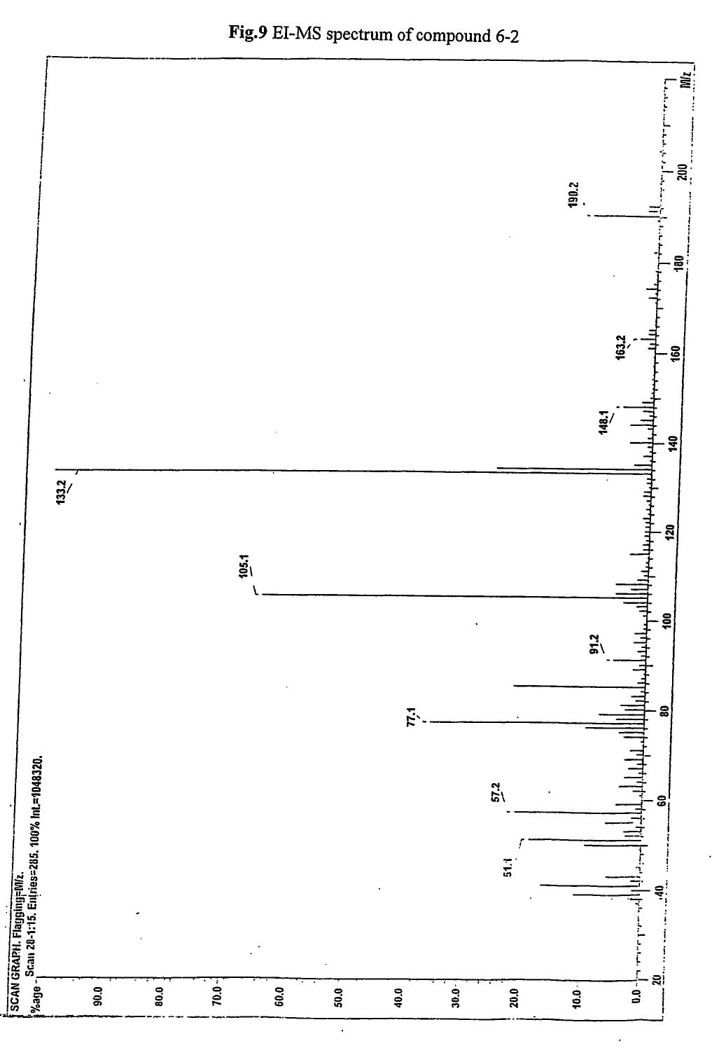

FIG. 9 shows the EI-MS spectrum of compound 6-1

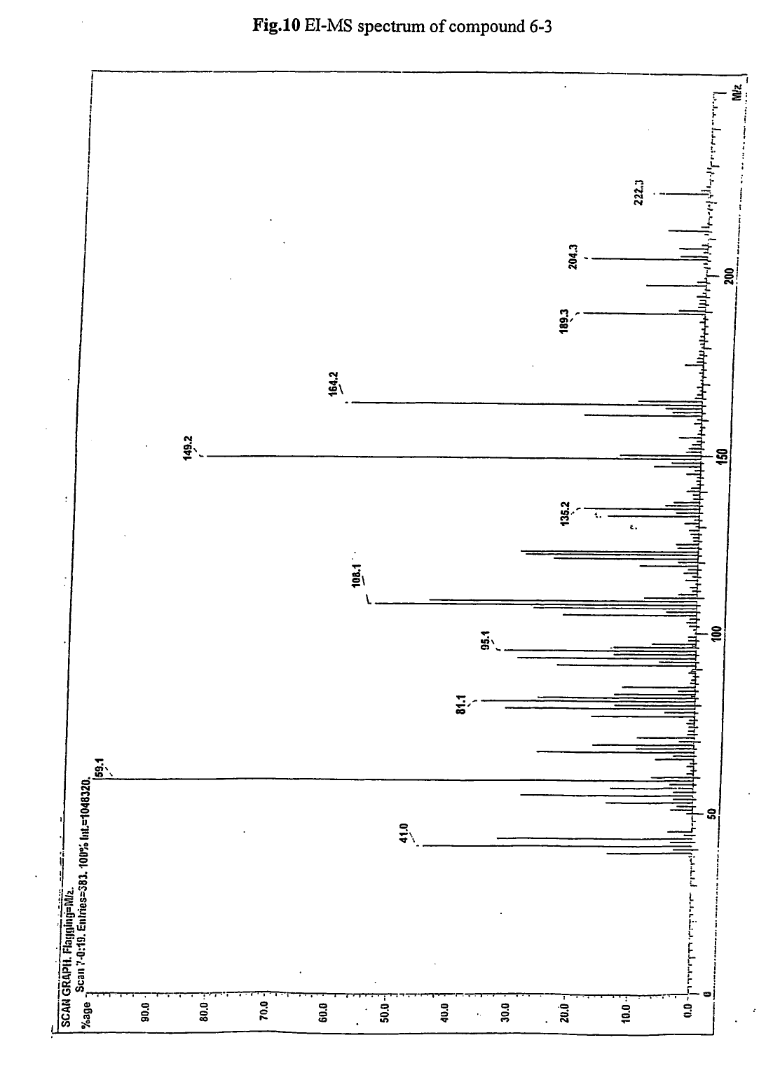

FIG. 10 shows the EI-MS spectrum of compound 6-3

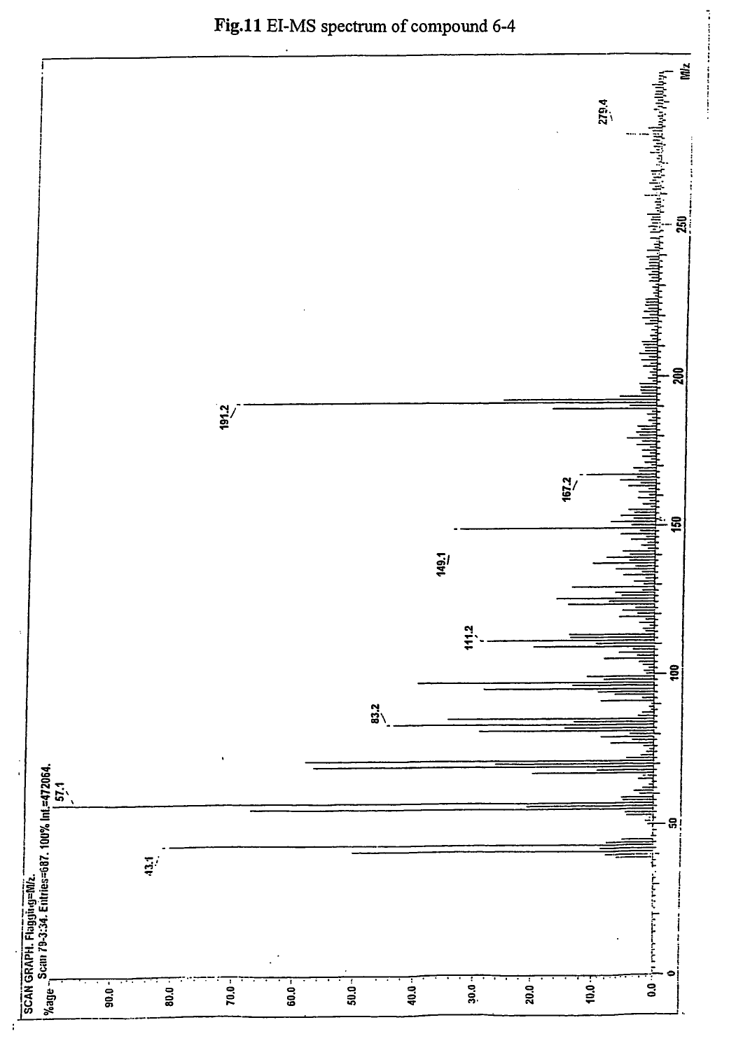

FIG. 11 shows the EI-MS spectrum of compound 6-4

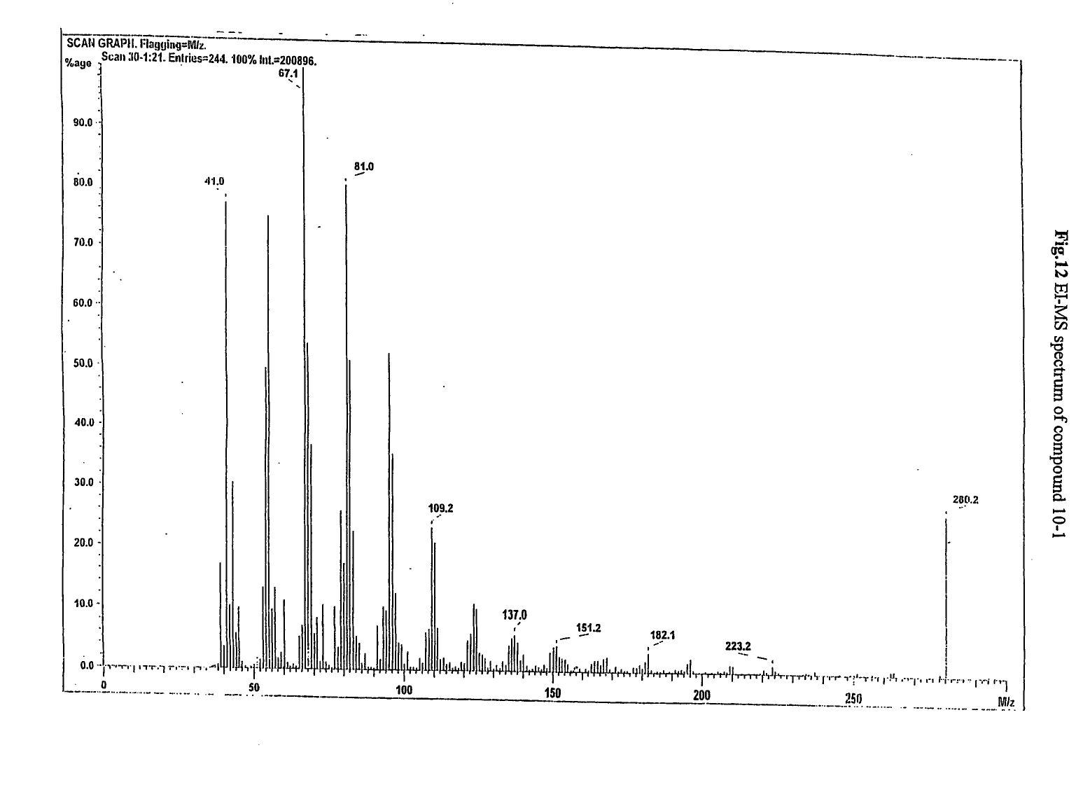

FIG. 12 shows the EI-MS spectrum of compound 10-1

Antimicrobial Test

Bacterial Strains

Three strains of H. pylori (3330, 3336 and 3339) isolated from British patients with gastric ulcer (duodenal ulcer or gastritis) were studied. The identities of H. pylori were confirmed by Gram stain and urease reaction. The bacteria were stored at −80° C. in aliquots of 1 ml of brocella broth containing 15% (v/v) glycerol (Kitsos and Stadtiander, 1998).

Celery Seed Extract (CSE)

Test CSE was provided as dark green highly viscous liquid (supplied by Beagle International Pty. Ltd, Nerang, Qld., Australia). Initally CSE was dissolved in dimethylsulfoxide (DMSO) as stock solution (100 mg/ml, final DMSO concentration in cultures ≦1%).

Media

For the Brucella broth (BB), (BBL, USA), Brucella (28 g) was added to IL of distilled water. After the medium was autoclaved at 120° C. for 15 mins, fetal bovine serum (50 ml) was added (Morgan et al, 1987).

Inocula

Thawed isolates were inoculated onto chocolate agar plates (Mërieux) and incubated under microacroplJnllic conditions (85%N₂, 10%CO₂, 5%0₂) for 48 h at 37° C. Colonies were suspended in 5ml of Brucella broth and adjusted to a turbidity equivalent to a No.2 McFarland standard (approximately 6×10⁸ CFU/ml) for broth dilution method. The final inoculum was 10⁷ CFU/ml for agar dilution method by a further 50-fold dilution.

Broth Dilution Test

The CSE suspension (1 mg/ml) was serially two-fold diluted in BB. The concentrations (1000, 500, 250, 125, and 62.5 μg/ml) were obtained. The solutions were added to the column wells of 24-well plate each in. equal volume (1 ml/well). 20 μl of cell suspension was inoculated into each row wells of 24-well plates (except last row wells). The culture dishes were gently agitated following the addition of the inoculum and then placed at 37° C. under microaeophilic conditions for three days. At the end of incubation, 1 ml of bacterial culture solution from each well were diluted to one in a million dilution (10⁻⁶). Then 20 μl aliquots from each solution were transferred to columbia agars and incubated for an additional three days. Generally, only spots with between 7-11 colonies were counted. Growth was determined on the basis of calculating the number of bacteria per milliliter (numbers of ba/ml=numbers of colonies on plate×reciprocal of dilution of sample). Bacteria growth, culture medium and extract controls were run in parallel. (Osato et al, 1999).

Chromatograplic Methods

Column chromatography was performed on silica- gel 60 (40-60 μm, Merck). Analytical thin layer chromatography (TLC) was carried out on precoated silica gel 60 F₂₅₄ plates (layer thickness 0.2 min, Merck), developed with the following solvent, hexane-EtOAc (70:30), chloroform-methanol (98:2). For isolation monitoring, spots were located by their absorption under ultraviolet (UV) light (254 and 366 nm) directly. After that the plates were sprayed with anisaldehyde reagent and heated at 110° C. for 5 min (Dey and Harbourne, 1991).

HPLC (1090 LC, Hewlett Packard, UK) Analytical and Semi-Preparative Purification

Analytical Conditions

Analytical column: Nucelosil® C18, particle size 5 μm, 250×4.6 mm I.D., catalogue No.89141 (Alltech, Carnforth, Lancashire, UK)

Mobile phase: acetonitrile/water (60:40)

Flow rate: 1.0 m/min

Injection volume: 10 μl

Detection: UV @236 nm

Sample: mixture of compounds 10-2, 10-3 and 10-4 (Conc.=5 mg/ml)

Temperature: ambient

ATT: 3

Semi-preparative conditions:

Semi-preparative column: Luna C18(2), particle size 5 μm, 250×10.00 mm I.D., catalogue No.00G-4252-NO (Phenomenex, Macclesfield, Cheshire, UK)

Mobile phase: acetonitrile/water (60:40)

Flow rate: 5.0 ml/min

Injection volume: 100 μl

Detection: UV @ 236 nm

Sample: mixture of compounds 10-2, 10-3 and 10-4 (Cone. 5 mg/ml)

Temperature: ambient

ATT: 6

Spectroscopic Methods

Mass spectrometry (MS)

The Mass spectra were recorded on a VG 70/70 Sector Mass Spectrometer instrument (Micromas, Manchester, UK) in the Laboratory of Biomedical research center (Sheffield Hallam University).

Nuclear magnetic resonance (NMR)

NMR spectra were recorded in CDCl₃ at RT on a Bruker Unity Ac 250 MHz (¹H 250 MHz; ¹³C, 62.9 Mhz).

### Results and Discussion

The 80%/ ethanol extract exhibited appreciable antimicrobial activity at the minimum inhibitory concentrations (MIC) of 250, 125 and 125 μg/ml, respectively, against H. pylori strains 3330, 3336 and 3339. The results of antimicrobial activity of CSE are given in Table 1 and FIG. 1. The bioassay-guided frationation scheme of CSE is illustrated in FIG. 2. The frationation for the isolation of the active compounds was performed from the 80% ethanol extract of CSE. The susceptibility of H. pylori strain 3339 was higher than 3330 and 3336. Later, in antimicrobial activity testing of fractions and subfractions of CSE, only H. pylori 3339 strain was chosen for fractionation guide. The residue of 80% ethanol extract of CSE was subsequently successively partitioned with organic solvents and water. The activity emerged predominantly in the petroleum ether layer (MIC-15.625 μg/ml) as compared to the other solvents, diethyl ether (MIC=125 μg/ml), ethyl acetate (MIC>500 μg/ml) and water (MIC>500 μg/ml) (Table 2).

The petroleum ether fraction was directly subjected to colt chromatography on silica gel with hexane, hexane-EtOAc (99:1), hexane-EtOAc (95:5) hexane-EtOAc (70:30) and EtOAc as eluent. Fractions with the same retardation factors were combined to yield 11 major fractions. Each subfraction was tested for antibacterial activity against H. pylori. The results of the antimicrobial testing of the different subfractions are shown in Table 3. The most pronounced antimicrobial activity successively resided in the subfraction 6 eluted with hexane-EtOAc (95:5) (MIC=15.625 μg/ml) and the subfraction 10 eluted with hexane-EtOAc (70:30) (MIC=15.625 μg/ml (FIG. 3). Subfraction 6 was further purified by silica gel column chromatography (hexane-ether, 10:1, as solvent) and preparative TLC using chloroform/pet. ether (3:1) to yield compounds 6-1, 6-2, 6-3 and 6-4. Subfraction 10 was further purified with hexane-ether (7:3) as mobile phase to afford a pure compound 10-1 and a mixture. The mixture was dissolved in 40% ACN in water and passed through the DPA-6S SPE column (Supelco, UK) to remove the chlorophyll. The eluate with methanol was evaporated to dryness and reconstituted in 40% ACN in water for HPLC analysis. It was separated into three compounds 10-2, 10-3 and 10-4 by analytical HPLC using ACN/water (60:40) as mobile phase (FIG. 4). Large quantity of individual pure compounds will be obtained by semi-preparative HPLC and sent for MS and NMR spectroscopic analysis.

Compounds 6-1, 6-2, 6-3, 10-1 and the combination of 6-1 and 6-3 were evaluated for antimicrobial activity. The results indicated they were partly responsible for the antimicrobial activity of CSE (Table 4 and FIG. 5). The mixture of 6-1 and 6-3 by different combination did not exert a syngergistic effect in antimicrobial activity. The mixture of compounds 10-2, 10-3 and 10-4 showed an interesting antimicrobial activity against H. pylori. Very recently, Momin and Nair (2001) isolated and characterized three bioactive compounds, sedanolide, senkyuolide-N and senkyunolide-J from CSE with the significant mosquitocidal, nematicidal and antifungal activities. Further study will confirm with MS and NMR data if compounds 10-2, 10-3 and 10-4 are corresponding to sedanolide, sekynuolide-N and sekyunolide-J. The antimicrobial activity of individual compound will be tested as well.

The exact structures are confirmed by comparison of their physical and spectral data ([α], 1H and 13NMR) with data in the literature. Structural elucidation of the compounds isolated from active fractions 6 and 10 are given below:

Compound 6-1 was obtained as pale yellow oil with a distinct celery odour. The electron impact mass spectrometry (EI-MS) spectrum (FIG. 6) of the compounds showed the molecular ion peak at mass/charge ratio (m/z) 192 (composition, 22.9%), corresponding to the molecular formula C₁₂_{H}₁₆0₂. Other major peaks were at m/z (composition, %) 163 (3.6), 135 (5.3), 10B (21.7), 107 (100%), 85 (9.7), 79 (24.3), 77 (24.2) and 57 (14.4).

The ¹H NMR spectrum (FIG. 7) displayed a doublet at 6.12 ppm (1H, J=10 Hz) and a multiplet at 5.9 ppm for the vinyl protons, H-7 and H-6, respectively, as well as multiplet at 4.9 ppm for H-3. In ¹³C NMR spectrum (FIG. 8), the signals at 128.4, 116.8 and 124.5 ppm were consistent with disubstituted and tetrasubstituted double bands composed of C-6, C-7 and C-1a, C-3a, respectively. In addition, tetra substituted signals appeared for the side chain (C-1′, C-2′, C-3′, C-4′) in the range of 13.8-22.4 ppm. The signals due to C-1, CA and C-S appeared at 161, 31.9 and 26,7 ppm.

On the basis of EI-MS and ¹H- and ¹³C-NMR, compound 6-1 was identified as 3-n-butyl 4,5-dihydrolphthalide (sedanenolide) (Bjeldanes and Kim, 1977).

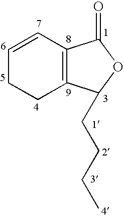

### Experimental Data

Compound 6-1 EI-MS: m/Z 192.3 (calculated for C₁₂H₁₆O₂). ¹H NMR (CDCl₃): δ 0.9 (t, 3H, J=7.2, H-4′), 1.2-1.8 [m, 6H, H1(1′, 2′, 3′)], 2.45 (m, H-4,5), 4.9 (m, 1H, H-3), 5.9 (m, 1H, H-6), 6.2 (d, 1H, J=10, H-7); ¹³C NMR (CDCl₃): δ 13.8-22.4 (C-1′, 2′, 3′, 4′), 26.7-31.8 (C-4.5), 82.5 (C-3), 116.8 (C-7), 128.3 (C-6), 124.5-135 (C-8, 9), 161.4 (C-1).

Compound 6-2 was obtained as pale yellow oil with a distinct celery colour. The EI-MS spectrum (FIG. 9) of 6-2 showed the molecular ion peak as mass/charge ratio (m/z) 190, Corresponding to the molecular formula C₁₂H₁₄O₂. Other major peaks were at m/z 163, 148, 144. 133 (100%), 115, 105, 91 and 77.

On the basis of EI-MS and ¹H- and ¹³C-NMR, compound 6-2 was identified as 3-n-butyl phthalide (Zheng et al 1993).

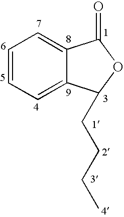

### Experimental Data

EI-MS: m/z 190.2 (calculated for C₁₂H₁₄O₂). ¹H NMR (CDCl3): δ 0.85 (t, 3H J=7.1, H-4′), 1.2-2.10 (m, 6H, H-(1′ 2′, 3′), 5.42 (dd, 1H, J=7.8 and 4.1 Hz, H-3), 7.39 (d. 1H, J=7.5, H4), 7.46 (t, 1H, J=7.5, H-6), 7.62 (t, 1H, J=7.5 HZ, H-5), 7.83 (d, 1H, J=7.5 Hz, H-7); ¹³C NMR (CDCL₃): δ14.08 (C-4′), 22.65 (C-3′), 27.01 (C-1′), 34.62 (C-2′), 81.75 (C-3), 121.68 (C-4), 125.57 (C-6), 125.96 (C-9), 128.94 (C-7), 134.20 (C-5), 150.02 (C-8), 171.04 (C-1).

(Large quantity of 62 will be obtained by purification using PTLC or semi-preparative HPLC, then ¹H NMR and ¹³C NMR will be acquired again to get clear spectra).

For compound 6-3, the EI-MS spectrum (FIG. 10) showed the molecular ion peak at mass/charge ratio (m/z) 222, corresponding to the molecular formula C₁₅H₂₆O. Other major peaks were at m/z 204, 189, 162, 149, 135, 109, 108, 95, 81, 59 and 41. On the basis of EI-MS, the compound 6-3 was identified as mixture of α and β-Eudesmol (El-Sayed et al. 1989).

¹H NMR and ¹³C NMR spectra will confirm the structure of 6-3. But there is not enough sample by now for measuring ¹H NMR and ¹³NMR (around 10-20 mg needed). The possible structure of compound 6-3 is as below:

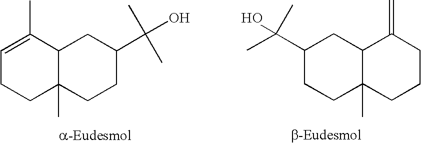

Compound 6-4 was obtained as colorless oil The EI-MS spectrum of 64 (FIG. 11) showed tho major peaks at m/z 279, 167, 149, 83, 71, 57 and 43, On the basis of EI-MS, the Compound 64 was identified as dioctyl phthalate, corresponding to tire molecular formula C₂₄H₃₈O₄ (MW=390.54 ) (MS library).

¹H NMR and ¹³C NMR spectra will confirm the structure of 64. But there is not enough sample by now for measuring ¹H NMR and ¹³C NME (around 10-20 mg needed). The possible structure of compound 6-4 is as below:

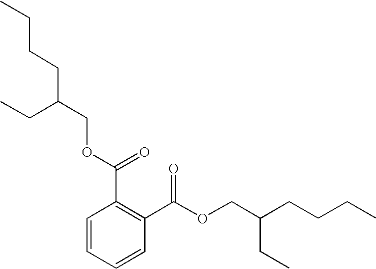

Compound 10-1 was obtained as a colourless oil. The EI-MS spectrum (FIG. 12) of 10-1 showed the molecular ion peak at mass/charge ration (m/z) 280, corresponding to the molecular formula C₁₈H₃₂O₂. Other major peaks were at m/z 137, 123, 109, 95, 81, 67, 55, 54 and 41. On the basis of EI-MS, the compound 10-1 was identified as linoleic acid (cis, cis - 9,12- Octadecadienoic acid) (MS library).

¹H NMR and ¹³C NMR spectra will confirm the structure of 10-1. But there is not enough sample for measuring ¹H NMR and ¹³C NMR (around 10-20 mg).

The possible structure of compound 10-1 is as below:

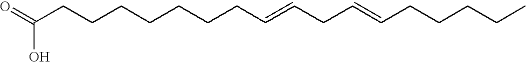

### CONCLUSION

Overall the CSE has showm interesting antimicrobial activity against H. pylori. Five compounds have been purified which arc partly responsible for the antimicrobial properties. The structure elucidation of compounds is still undergoing. Further work will continue to purify the active constituents in subfraction 10 and other subtractions and to test the anti-cytokine activity and cartilage protection properties. If the compounds from subfractions 6 and 10 are not responsible for the anti-inflammator activity, the constituents maybe reside in other fractions and subfractions.

### REFERENCES

Bjeldanes L. F. and KIM I. S. (1977) Phthalide components of celery essential oil. J. Org. Chem. 42(13),23333-5.
Chan. F. K. L., Sung J. Y., Leung V. K. S. et al (1997) Randomized trial of eradication of H. pylori before non-steroid anti-inflammatory drug therapy to prevent peptic ulcer. Lancet 350, 975-9.
Dey P. M. and Harborne J. B. (1991) Methods in plant Biochemistry, volume 7, Terpenoids, Edited by Charlwood B. V. and Banthorpe D. V., Academic Press, p.65.
El-Sayed A. M. Al-Yahya M. A. Hassan, M. M. (1989) Chemical composition and antimicrobial activity of the essential oil of Chenopodium botrys growing in Saudi Arabia. Int. J. Crude Drug Res. 27, 185-188.
Kitsos C. M. and Stadtländer C. T., (1998) Helicobacter pylori in liquid culture: Evaluation of growth rates and ultrastructure. Curr. Microbiol. 37, 88-93.
Lewis D. A., Darib S. M. and Veitch G. B. A. (1985). The anti-inflammatory activity of celery Apium graveolens L. (Fam. Umbelliferae) Int J. Crude Drug Res. 23, 27-32.
Momin R. A. and Nair M. G. (2001) Mosquitocidal, and antifungal compounds from Apium graveolens L. seeds. J. Agric. Food Chcm. 49, 142-145.
Morgan D., Freedman R., Depew C., and Kraft W. (1987) Growth of Campylobacter pylori in liquid media. J. Clin. Microbiol. 25,2123-2125.
Osato M. S., Reddy S. G. and Graham, D. Y. (1999) Osmotic effect of honey on growth and viability of H. pylori . Dig. Dis. Sci. 44, 462-464.

Zheng G. Q.; Kenney P. M.; Zhang J.; Lam L. K. T. (1993) Chemoprevention of benzo[a]pyrene-induced forestomach cancer on mice by natural phthalides from celery seed oil. Nutr. Cancer 19(1), 77-86.

TABLE 1
Effect of the crude extract of CSE on the growth of different
strains (3330, 3336 and 3339) on H. pylori.

| Strains | MIC (μg/ml) | MBC (μg/ml) |
|---|---|---|
| 3330 | 250 | 500 |
| 3336 | 125 | 500 |
| 3339 | 125 | 500 |

TABLE 2
Distribution of antimicrobial activty against H. pylori (strain
3339) in the crude extract and different fractions of CSE.

| Fractions | MIC (μg/ml) | MBC (μg/ml) |
|---|---|---|
| Crude extract | 125 | 500 |
| Pet. ether | 15.625 | 31.25 |
| Diethyl ether | 125 | 500 |
| Ethylacetate | >500 | >500 |
| Water | >500 | >500 |

TABLE 3
Antimicrobial activity of the subfractions from pet.
ether fraction against H. pylori (strain 3339).

| Fractions and subfractions | MIC (μg/ml) |
|---|---|
| Pet. ether | 15.625 |
| Sub-1 | >125 |
| Sub-2 | >125 |
| Sub-3 | 125 |
| Sub-4 | 62.5 |
| Sub-5 | 62.5 |
| Sub-6 | 15.625 |
| Sub-7 | 31.25 |
| Sub-8 | 31.25 |
| Sub-9 | 62.5 |
| Sub-10 | 15.625 |
| Sub-11 | 31.25 |

TABLE 4
Antimicrobial activities of compounds from subfractions
6 and 10 against H. Pylori (strain 3339).

| Compounds | MIC (g/ml) | MBC (g/ml) |
|---|---|---|
| sedanenolide | 31.25 | 62.5 |
| 3-n Butyl phthalide | 15.625 | N.T. |
| Eudesmol | 15.625 | 125 |
| Eudesmol + sedanenolide | 15.625 | N.T. |
| (major) (minor) | | |
| Eudesmol + sedanenolide | 31.25 | N.T. |
| (minor) (major) | | |
| Linoleic acid | 62.5 | >125 |
| 10-2, 10-3 and 10-4 | 12.5 | 25 |

N.T.: not tested

1. Use of celery seed or celery seed extract (CSE) for the inhibition of growth and replication of Helicobacter pylori.

2. Celery seed or celery seed extract for the preparation of a pharmaceutical composition to treat Helicobacter pylori infection.

3. Use according to claim 1 or claim 2 wherein the celery seed extract is an ethanol/water extract.

4. Use according to any preceding claim wherein the active component of the celery seed extract is selected from 3-n-butyl 4,5dihydrolphthalide, 3-n-butyl phthalide, α-Eudesmol, β-Eudesmol dioctyl phthalate and cis, cis-9,12-Octadecadienoic acid.

5. A method of treating Helicobacter pylori infection comprising administering a pharmaceutically effective amount of celery seed or a celery seed extract
